(19) United States
(12) Patent Application Publication
(10) Pub. No.: US 2025/0302440 A1
(43) Pub. Date: Oct. 2, 2025

(54) Method and System for Acoustic Crosstalk Suppression

(71) Applicant: BFLY Operations, Inc, Burlington MA, MA (US)

(72) Inventors: Simon Esteve, Guilford, CT (US); Jaime Scott Zahorian, Guilford CT, CT (US); Farah Qureshi, Sunnyvale, CA (US); Sarp Satir, Branford, CT (US); Timothy Hyde, Columbia, SC (US); Scott Rumschlag, Burlington MA, MA (US)

(73) Assignee: BFLY Operations, Inc, Burlington MA, MA (US)

(21) Appl. No.: 18/864,348

(22) PCT Filed: May 9, 2023
(86) PCT No.: PCT/US2023/021497

§ 371 Date: Nov. 8, 2024

Related U.S. Application Data
(60) Provisional application No. 63/339,928, filed on May 9, 2022.

(51) Int. Cl. A61B 8/00; B06B 1/02

(52) U.S. Cl. CPC A61B 8/4477; A61B 8/4272; B06B 1/0292; A61B 8/4427; B06B 2201/76

(57) ABSTRACT

An ultrasound probe includes an ultrasound transducer stack, an acoustic lens, and an acoustic coupling layer between the acoustic lens and the ultrasound transducer stack. The transducer stack includes one or more ultrasound transducers emitting an acoustic signal and the acoustic lens focuses the acoustic signal. The acoustic coupling layer has a speed of sound that is higher than a speed of sound in the acoustic lens, and the acoustic coupling layer has a thickness between a quarter and half a wavelength of the acoustic signal.

### CROSS-REFERENCE TO RELATED APPLICATIONS

This application claims the benefit of priority under 35 U S.C. § 119 (e) to U.S. Provisional Patent Application Ser. No. 63/339,928, filed on May 9, 2022, which is hereby incorporated by reference herein in its entirety.

### BACKGROUND

An ultrasound probe may include multiple ultrasound transducers arranged in a transducer array that emits ultrasound signals. The ultrasound signals may be reflected by body tissue thereby resulting in an echo. The ultrasound transducers may receive the echo as a received ultrasound signal, and the received ultrasound signal may be processed to generate an ultrasound image or sonogram.

The sonogram may suffer from acoustic crosstalk between the individual ultrasound transducers in the transducer array of the ultrasound probe. For example, acoustic crosstalk may cause resonances resulting in excessive ringing of individual ultrasound transducers of the transducer array. This may cause noise on the signal obtained from the transducer array. Acoustic crosstalk may further cause individual ultrasound transducers to operate in unwanted higher order resonant modes, which may cause damage. A crosstalk signal from neighboring ultrasound transducers may further cause a transducer array of the ultrasound probe to be more sensitive at certain angles and less sensitive at others, depending on whether there is constructive of destructive interference between adjacent ultrasound transducers in the transducer array. In view of these issues, it may be desirable to suppress or at least reduce acoustic crosstalk between individual ultrasound transducers of the transducer array.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to an ultrasound probe, comprising: an ultrasound transducer stack comprising one or more ultrasound transducers emitting an acoustic signal; an acoustic lens focusing the acoustic signal; and an acoustic coupling layer between the acoustic lens and the ultrasound transducer stack, wherein the acoustic coupling layer has a speed of sound that is higher than a speed of sound in the acoustic lens, and wherein the acoustic coupling layer has a thickness between a quarter and half a wavelength of the acoustic signal.

In general, in one aspect, embodiments relate to an ultrasound probe, comprising: an ultrasound transducer stack comprising one or more ultrasound transducers emitting an acoustic signal; an acoustic lens focusing the acoustic signal; and an acoustic coupling layer between the acoustic lens and the ultrasound transducer stack, wherein the acoustic lens comprises at least one standoff that defines a space for the acoustic coupling layer between the acoustic lens and the ultrasound transducer stack.

In general, in one aspect, embodiments relate to a method of manufacturing an ultrasound probe, the method comprising: depositing an acoustic coupling layer between an ultrasound transducer stack comprising one or more ultrasound transducers for emitting an acoustic signal and an acoustic lens for focusing the acoustic signal, wherein the acoustic lens comprises at least one standoff that defines a space for the acoustic coupling layer between the acoustic lens and the ultrasound transducer stack.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows an ultrasound imaging scenario in accordance with one or more embodiments.

FIG. 2A schematically shows a cross-sectional view of an ultrasound probe with acoustic crosstalk attenuation in accordance with one or more embodiments.

FIGS. 2B and 2C schematically show acoustic wave propagation in an ultrasound probe with acoustic crosstalk attenuation in accordance with one or more embodiments.

FIGS. 3A, 3B, and 3C show different views of elements of an ultrasound probe in accordance with one or more embodiments.

FIG. 4 schematically shows an implementation example of an ultrasound system integrated on a chip, in accordance with one or more embodiments.

FIGS. 5A and 5B show flowcharts of methods of manufacturing an ultrasound probe in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for reducing or suppressing acoustic crosstalk between individual elements (e.g., ultrasound transducers) in a transducer array of an ultrasound probe. An ultrasound transducer array may be equipped with an acoustic lens. The acoustic lens may couple the acoustic energy to and from the ultrasound transducers and focus the acoustic energy onto a focal spot. When the acoustic lens is directly in contact with the transducer array, acoustic crosstalk may occur between the individual ultrasound transducers within the lens material. In one or more embodiments, the acoustic crosstalk is reduced or suppressed by an acoustic coupling layer or boundary layer between the transducer array and the acoustic lens.

The reduced or suppressed acoustic crosstalk may provide one or more of the following benefits. The quality of the signal obtained from the transducer signal may improve, due to a reduction or elimination of the acoustic crosstalk. The sensitivity of the transducer array may be more uniform across an angular range, because possible constructive and destructive angle-dependent interference when steering the acoustic beam may be reduced or eliminated. The robustness and/or longevity of the transducer array may be improved, because potentially damaging higher order resonant modes are avoided. A detailed description is subsequently provided.

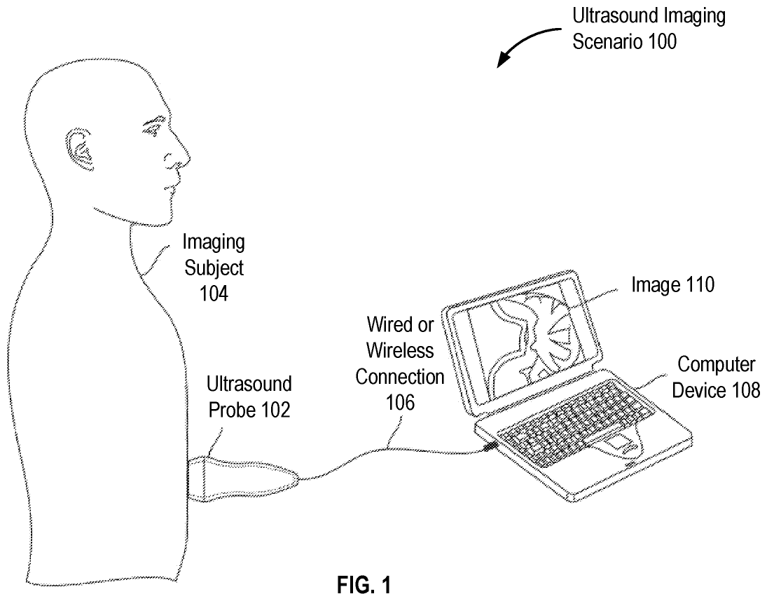

FIG. 1 shows an example of an ultrasound imaging scenario in accordance with one or more embodiments. The ultrasound imaging scenario (100) illustrates the use of an ultrasound probe (102) to obtain ultrasound images (sonograms) from an imaging subject (104). Data collected by the ultrasound probe (102) may be transmitted to one or more external computer devices (108) for further processing. For example, ultrasound probe (102) may transmit the data via a wired or wireless connection (106) to a computer device (108) (a laptop in this non-limiting example), which may process the data to generate and display an image (110) of the imaging subject (104) on a display.

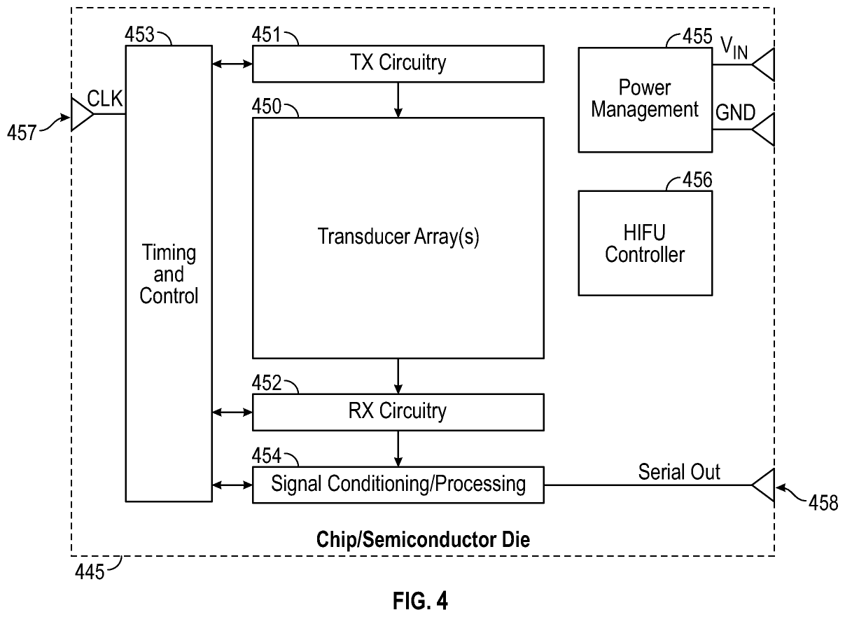

The ultrasound probe (102) may include various components that enable the transmission and/or reception of acoustic waves, as subsequently discussed. The components may be arranged in different manners, without departing from the disclosure. For example, various components of the ultrasound probe (102) may be integrated on chip. Alternatively, discrete components of partially integrated components may be used. An example of a configuration that includes ultrasound transducers as well as ultrasound circuitry integrated on a chip is described below in reference to FIG. 4.

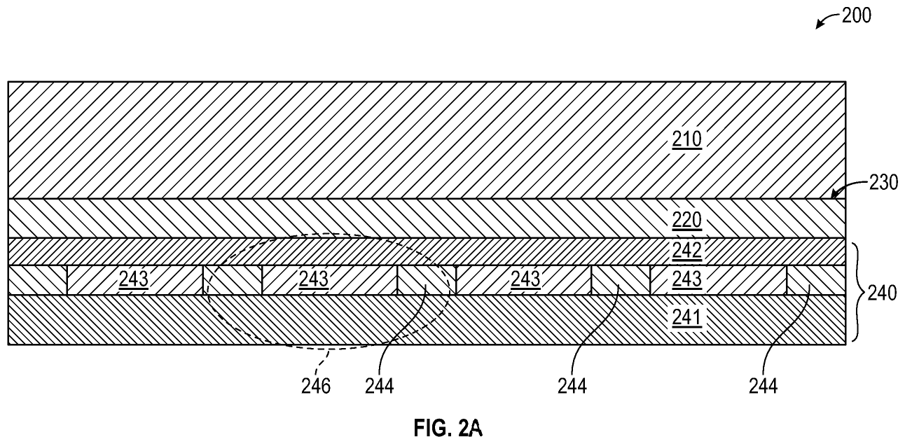

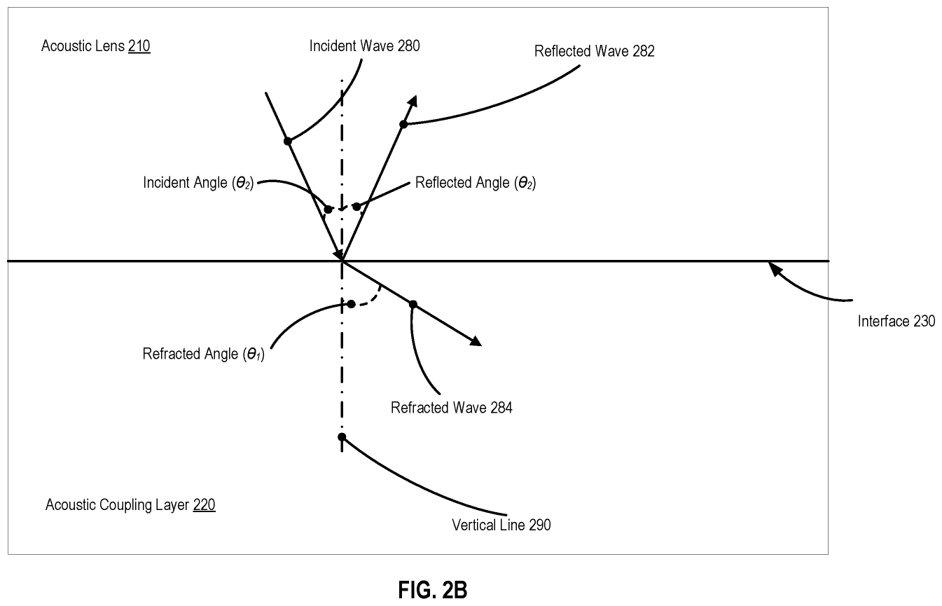

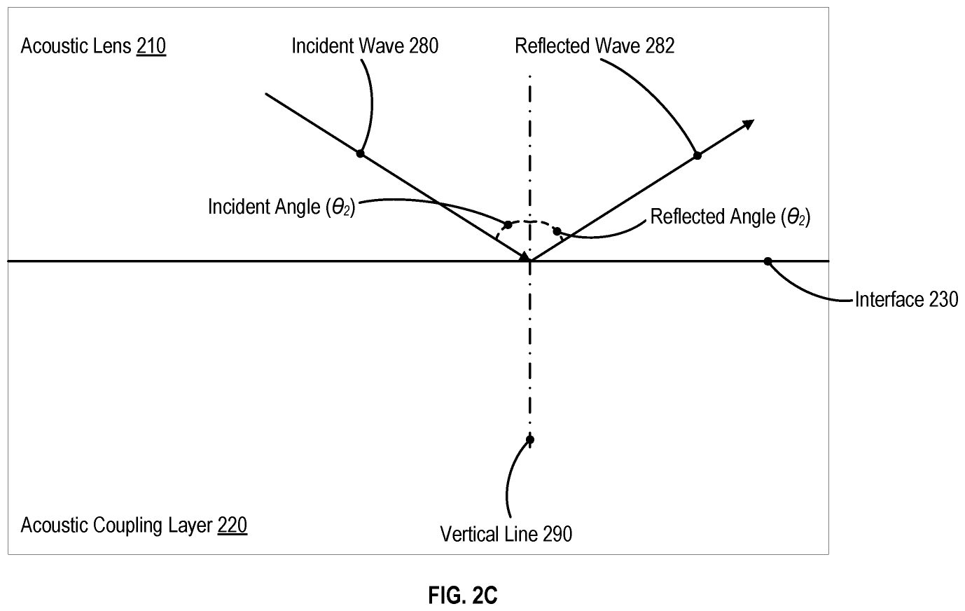

Turning to FIGS. 2A, 2B, and 2C, FIG. 2A shows a simplified cross-sectional view of an ultrasound probe with acoustic crosstalk attenuation (200) in accordance with one or more embodiments. As shown in FIG. 2A, an ultrasound probe with acoustic crosstalk attenuation (200) may include an acoustic coupling layer (220) (e.g., a boundary layer) embedded between ultrasound transducers (246) and an acoustic lens (210) to suppress the acoustic crosstalk that may occur between individual elements within the ultrasound probe. While FIGS. 2A, 2B, and 2C show certain elements, the ultrasound probe may include additional elements, without departing from the disclosure. In one or more embodiments, the ultrasound transducers (246) are formed by elements arranged in an ultrasound transducer stack (240). The ultrasound transducers may be arranged in a transducer array which may be integrated on a single semiconductor die. In the example shown in FIG. 2A, the transducer stack (240) includes a substrate (241), a membrane (242), and cavity sidewalls (244) which enclose cavities (243). In the area of each of the cavities (243), the membrane (242) may vibrate, thus forming an ultrasound transducer (246). The ultrasound transducers (246) may be used to transduce an acoustic signal into an electric signal, or vice versa. Silicon materials may be used for the substrate (241), the membrane (242), and/or the cavity sidewalls (244), and the ultrasound transducers (246) may be on a chip.

In one or more embodiments, the ultrasound transducers (246) formed in the ultrasound transducer stack (240) are Capacitive Micromachined Ultrasonic Transducers (CMUTs) in which the cavities (243) are micromachined. A more detailed description may be found in, for example, U.S. Pat. No. 9,067,779, and U.S. patent application Ser. No. 16/296,476 which are hereby incorporated by reference in their entirety. While not shown, the substrate (241) may also accommodate integrated circuitry used for driving and/or interrogating the ultrasound transducers (246).

Also, the transducer stack (240) may include other components, e.g., a heat spreader for cooling the chip with the transducers, a printed circuit board that accommodates the chip with the transducers, etc.

In one or more embodiments, the acoustic coupling layer (220) provides a thin boundary layer of a material (such as a silicone, epoxy (e.g., Loctite Stycast 1265), etc.) with high acoustic attenuation (e.g., an attenuation of 40-200 dB/cm at 5 MHz) to further suppress acoustic crosstalk based on certain characteristics of the acoustic coupling layer (220), including a speed of sound c₁, density p₁, thickness Z₁, and attenuation factor Attn₁. In particular, in one or more embodiments, the acoustic coupling layer (220) has a speed of sound c₁ higher than the speed of sound c₂ of the acoustic lens (210) (c₁>c₂). The acoustic coupling layer (220) may further have an acoustic impedance that substantially matches (e.g., c₁p₁=c₂p₂) that of the acoustic lens (210) to minimize acoustic reflections at the interface (230) between the acoustic coupling layer (220) and the acoustic lens (210).

In absence of the acoustic coupling layer (220), acoustic crosstalk between individual elements (e.g., ultrasound transducers (246), etc.) may occur within the acoustic lens material. In one or more embodiments, the acoustic coupling layer (220), disposed between the ultrasound transducers (246) and the acoustic lens (210), reduces or eliminates the acoustic crosstalk.

The following discussion in reference to FIGS. 2B and 2C describes the effect of the interface (230) between the acoustic lens (210) and the acoustic coupling layer (220) on acoustic signals, using an illustration of how a single acoustic wave is affected.

FIG. 2B shows an incident acoustic wave (280) with an angle of incidence θ₂ in the acoustic lens (210) arriving at the interface (230) between the acoustic lens (210) and the acoustic coupling layer (220). The incident acoustic wave associated with acoustic crosstalk results in acoustic refraction at the interface (230) between the acoustic lens (210) and the acoustic coupling layer (220) when the incident acoustic wave travels from one medium into another based on Snell's law (Equation 1). The refracted acoustic wave (284) may have an angle θ₁ (Equation 2). Likewise, the reflected acoustic wave (282) may have an angle of θ₂. The incident/reflected/refracted angles are measured with respect to the vertical line (290) which is normal to the interface (230).

$$\frac{sin {θ}_{1}}{{c}_{1}} = \frac{sin {θ}_{2}}{{c}_{2}} \qquad ( 1 )$$

$${θ}_{1} = {sin}^{- 1} ( \frac{{c}_{1} sin {θ}_{2}}{{c}_{2}} ) \qquad ( 2 )$$

where θ₁ is angle of refraction of the acoustic wave in the acoustic coupling layer, θ₂ is angle of incidence of the acoustic wave in the acoustic lens, c₁ is speed of sound for the acoustic coupling layer, and c₂ is speed of sound for the acoustic lens.

In other words, the difference in speed of sound and acoustic impedance between acoustic coupling layer (c₁, c₁p₁) and the acoustic lens (c₂, c₂p₂) may cause acoustic refraction at the interface (230) between the acoustic coupling layer (220) and the acoustic lens (210) when sound travels from one medium into another. Importantly, if c₁, associated with the acoustic coupling layer (220), is greater than c₂, associated with the acoustic lens (210), then for a critical angle of incidence θ_{c} in the acoustic lens, the refracted acoustic wave (284) has an angle θ₁ which approaches 90 degrees) (Equation 3).

$${θ}_{c} = {sin}^{- 1} ( \frac{{c}_{2}}{{c}_{1}} ) \qquad ( 3 )$$

where θ_{c} is the critical angle, c₁ is a speed of sound for the acoustic coupling layer, and c₂ is a speed of sound for the acoustic lens.

FIG. 2C shows an acoustic wave travelling in the acoustic lens (210) incident at the interface (230) where it may experience total internal reflection if its angle of incidence exceeds a critical angle θ_{c} which depends on c₁ and c₂. For example, the reflected acoustic wave (282) may have an angle θ₂. There is no refracted wave (i.e., total internal reflection) because the conditions of refraction are not satisfied. As a result, the transmission across the interface (230) of acoustic waves that travel substantially laterally (thereby exceeding the critical angle θ_{c}) in the acoustic lens (210) may be impaired or blocked. The transducer elements may, thus, be shielded from acoustic crosstalk. Preferably the speed of sound in the acoustic coupling layer (220) should be >50% higher than in the acoustic lens (210) (i.e., c₁>>c₂). A smaller speed difference may be sufficient, though. For c₁>>c₂, the critical angle is smaller than for c₁>c₂. A smaller critical angle is preferred because it may provide a more reliable reduction of crosstalk, but larger critical angles may be acceptable.

In contrast, acoustic waves traveling in the acoustic coupling layer (220) incident at the interface always get transmitted into the acoustic lens (210), irrespective of the angle of incidence because c₁>c₂.

Acoustic crosstalk waves decrease in magnitude exponentially as they propagate away from the interface (230) because the acoustic crosstalk waves are evanescent. Therefore, a thin layer of attenuating material in the acoustic coupling layer (220) may be sufficient to suppress the acoustic crosstalk waves. Although the attenuating layer (e.g., the acoustic coupling layer (220)) may also suppress a desired acoustic wave, with the acoustic coupling layer (220) being sufficiently thin, the overall reduction in the desired acoustic waves may be minimal. The thickness of the acoustic coupling layer (220) should preferably be more than a quarter or less than half of the wavelength for the ultrasound frequency to be suppressed. This choice of the thickness of the acoustic coupling layer (220) relates to quarter wavelength and half wavelength of array resonances in the acoustic coupling layer (220) and may help avoid these array resonances. For example, for frequencies corresponding to most medical imaging applications, the thickness of the acoustic coupling layer (220) should be in the range of ˜75-200 micrometers (μm).

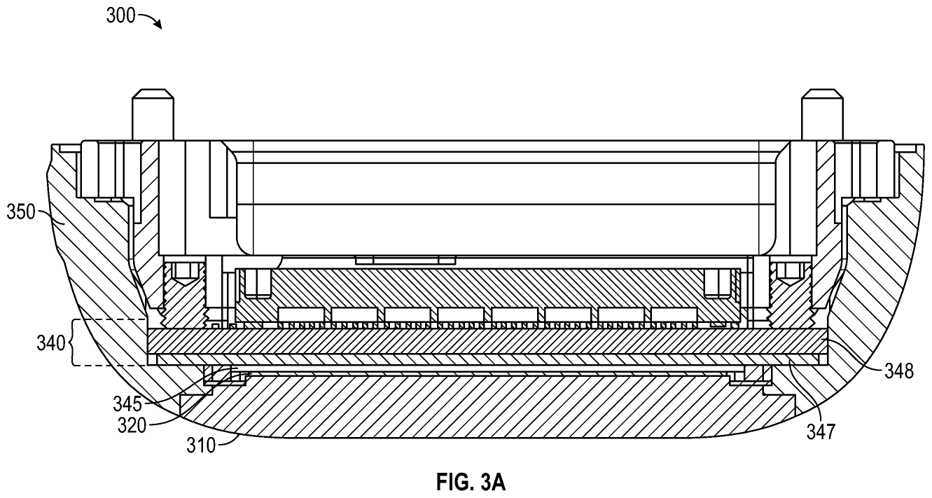

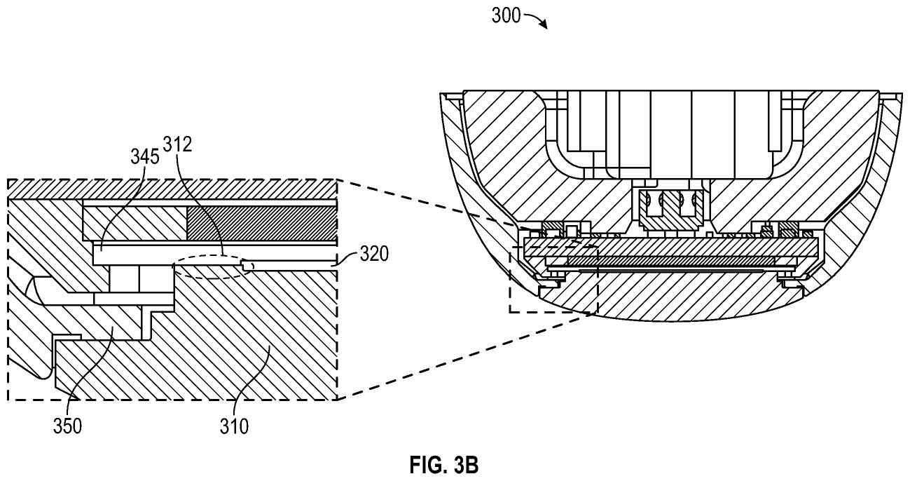

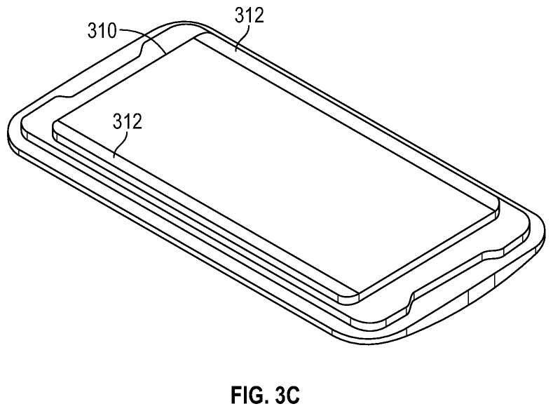

Turning to FIGS. 3A, 3B, and 3C, FIG. 3A shows an ultrasound probe in accordance with one or more embodiments. The ultrasound probe (300) includes a shroud (350), an ultrasound transducer stack (340), an acoustic coupling layer (320), and an acoustic lens (310). In the example of FIG. 3A, the ultrasound transducer stack (340) includes various elements such as the chip (345) with the ultrasound transducers, the heat spreader (347) and the printed circuit board (348), as previously described. While FIGS. 3A, 3B, and 3C show certain elements, the ultrasound probe may include additional elements, without departing from the disclosure. The shroud (350) houses the elements of the ultrasound probe (300) and may acoustically, thermally (e.g., acting as a heat sink), and/or mechanically (e.g., providing structural rigidity) protect the ultrasound transducer stack (340). The shroud (350) may be formed from the same material as the body of the ultrasound probe (300), e.g., aluminum, plastic, a composite material, etc.

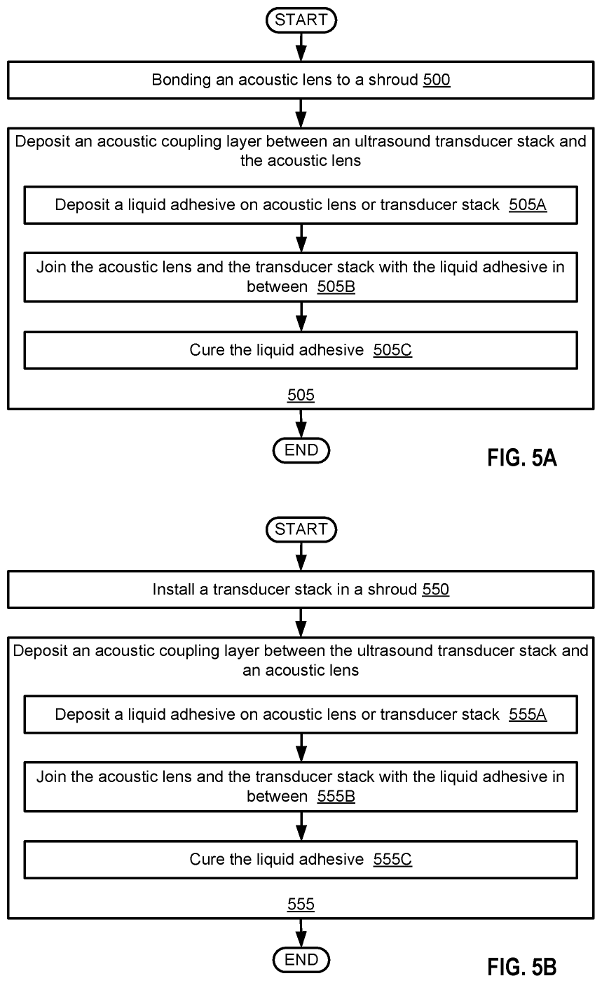

Each component of the ultrasound probe (300) may have a mechanical tolerance. In one or more embodiments, one or more of the components are designed such that the thickness of the acoustic coupling layer (320) does not exceed a certain value (e.g., 200 μm). For example, the thickness of the acoustic coupling layer (320) may be specified to be 0.1 mm+0.1 mm/−0.025 mm. After the acoustic coupling layer (320) is deposited on the acoustic lens (310), and the chip (345) is brought down (as further described in reference to FIGS. 5A-5B), features of the shroud (350) may ensure that the width of the acoustic coupling layer (320) does not exceed 200 μm, once the ultrasound probe (300) has been assembled.

FIGS. 3B and 3C provide additional views of elements of an ultrasound probe in accordance with one or more embodiments. A standoff (312) is added on the backside (facing the chip (345)) of the acoustic lens (310). The standoff (312) may be a raised portion or protrusion of the acoustic lens (310), establishing a defined space for the acoustic coupling layer (320) to enforce a thickness of no more than 200 μm. As shown in FIG. 3B, the standoff (312) may be in mechanical contact with an inactive area of the chip (345) (i.e., an area not involved in the emission/reception of acoustic waves). The standoff (312), thus, tightly controls the thickness of the acoustic coupling layer (320) between the acoustic lens (310) and the chip (345). In one example, the height of the standoff (312) is 0.090 mm +/−0.015 mm. In one or more embodiments, the standoff operates in conjunction with the acoustic lens (310) having a certain level of mechanical flexibility. Specifically, the standoff (312) may ensure that there is an acoustic coupling layer (320) of a specified thickness between the acoustic lens (310) and the chip (345), whereas the mechanical flexibility of the acoustic lens (310) ensures that the transducer stack (340) including the chip (345) is in a defined mechanical position relative to the shroud (350). In other words, during mechanical assembly of the ultrasound probe (300), the acoustic lens (310) may deform until the transducer stack (340) hard-stops on the shroud (350). The acoustic lens (310) may be made of any material suitable for providing desired lensing functionality, impedance matching, signal attenuation, and mechanical flexibility. Such materials include, but are not limited to room temperature vulcanizing silicone, rubber, etc.

FIG. 4 schematically shows an implementation example of an ultrasound system integrated on a chip (445), in accordance with one or more embodiments. The example is provided for illustrative purposes only and is not intended to limit the scope of the disclosure. The chip (445) may include one or more transducer arrangements (e.g., transducer array (450)), transmit (TX) circuitry (451), receive (RX) circuitry (452), a timing and control circuit (453), a signal conditioning/processing circuit (454), a power management circuit (455), and/or a high-intensity focused ultrasound (HIFU) controller (456). In the embodiment as shown, all of the illustrated elements are formed on a single semiconductor die. In other embodiments, one of more of the elements may be discrete components. In addition, although the illustrated example shows both TX circuitry (451) and RX circuitry (452), in alternative embodiments only TX circuitry (451) or only RX circuitry (452) may be employed. For example, such embodiments may be employed in transmission-only ultrasound probes or reception-only ultrasound probes. The TX circuitry (451) may generate pulses to energize the individual elements of the transducer array (450) so as to emit an ultrasound pulse for imaging. Likewise, the RX circuitry (452) may receive and process electronic signals generated by the individual elements of the transducer arrays (450). In one embodiment, the chip (445) accommodates the transducer array (450) on a plain substrate, whereas the other components shown in FIG. 4 are located elsewhere.

The ultrasound transducers in the transducer array (450) may be arranged in various manners. In some embodiments, the transducer array (450) may include capacitive micromachined ultrasonic transducers (CMUTs), CMOS ultrasonic transducers (CUTS), piezoelectric micromachined ultrasonic transducers (PMUTs), and/or other suitable ultrasonic transducer cells. The timing and control circuit (453) may generate various timing and control signals that may be used to synchronize and coordinate the operation of the components on the chip (445). An input port (457) may provide a clock signal CLK to supply the timing to the control circuit (453). The signal conditioning/processing circuit (454) may generate a high-speed serial data stream which is outputted by one or more output ports (458). The high-speed serial data stream may include the data (e.g., received acoustic signals) obtained from the transducer array (450) via the RX circuitry (452). The power management circuit (455) may convert one or more input voltages VIN from an off-chip source into voltages needed to carry out operation of the chip. Likewise, the power management circuit (455) may manage power consumption of the components on the chip (445).

The HIFU controller (456) may generate one or more HIFU signals via one or more elements of the transducer arrays (450) to provide HIFU functionality to provide the transducer arrays (450) a power level appropriate for imaging applications.

FIGS. 5A and 5B show flowcharts in accordance with one or more embodiments. FIGS. 5A and 5B describe methods for assembling an ultrasound probe. While the various blocks in FIGS. 5A and 5B are presented and described sequentially, one of ordinary skill in the art will appreciate that some or all of the blocks may be executed in different orders, may be combined or omitted, and some or all of the blocks may be executed in parallel.

Turning to FIG. 5A, in Block 500, an acoustic lens is bonded to a shroud, e.g., by gluing or overmolding. The acoustic lens may be flexible and may be equipped with one or more standoffs. Further details on the acoustic lens including the standoffs and the shroud may be found in FIGS. 3A, 3B, and 3C, and the accompanying description.

In Block 505, an acoustic coupling layer is deposited between an ultrasound transducer stack and the acoustic lens. In one or more embodiments, in Block 505A, a liquid adhesive, e.g., an epoxy is directly deposited onto the acoustic lens or onto the transducer stack, at the surfaces where the acoustic coupling layer is to be formed. In Block 505B, the acoustic lens and the transducer stack are joined with the liquid adhesive in between, resulting, for example, in the arrangements as shown in FIGS. 3A and 3B. The joining may be performed by lowering the transducer stack into position to come into contact with the liquid adhesive on the acoustic lens before the liquid adhesive is cured. Once in position, the transducer stack hard-stops on the shroud, with a defined thickness of the acoustic coupling layer being established by standoffs of the acoustic lens. The transducer stack may be secured (e.g., using screws). The lowering of the transducer stack may result in a deformation of the acoustic lens as the space between the transducer stack and the acoustic lens is reduced based on the height of the standoff(s). In Bock 505C, the liquid adhesive is cured to form the acoustic coupling layer in the space defined by the one or more standoffs. The type of curing may depend on the type of liquid adhesive. For example, the liquid adhesive may be left to cure at room temperature.

Turning to FIG. 5B, in Block 550, a transducer stack is installed in a shroud. The transducer stack may hard-stop on the shroud, with screws securing the transducer stack.

In Block 555, an acoustic coupling layer is deposited between the ultrasound transducer stack and an acoustic lens. In one or more embodiments, in Block 555A, a liquid adhesive, e.g., an epoxy is directly deposited onto the acoustic lens or onto the transducer stack, at the surfaces where the acoustic coupling layer is to be formed. In Block 555B, the acoustic lens and the transducer stack are joined with the liquid adhesive in between, resulting, for example, in the arrangements as shown in FIGS. 3A and 3B. The joining may be performed by lowering the acoustic lens into position with the liquid adhesive in contact with both the transducer stack and the acoustic lens. The lowering of the acoustic lens may result in a deformation of the acoustic lens as the space between the transducer stack and the acoustic lens is reduced based on the height of the standoff(s). Once in position with a defined thickness of the acoustic coupling layer established by the standoff(s), the acoustic lens is secured. Glue or overmolding may be used to secure the acoustic lens to the shroud. In Bock 555C, the liquid adhesive is cured to form the acoustic coupling layer in the space defined by the one or more standoffs. The type of curing may depend on the type of liquid adhesive. For example, the liquid adhesive may be left to cure at room temperature.

While the methods of FIGS. 5A and 5B describe the use of a liquid adhesive to form the acoustic coupling layer, a thin film (e.g., in the form of a double-sided tape) may be used instead. To ensure good acoustic performance, the installation of the thin film may involve steps to ensure that no air bubbles or other irregularities are present. Also, while not discussed, the methods of FIGS. 5A and 5B may include additional steps to complete the assembly, without departing from the disclosure.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

1. An ultrasound probe, comprising:
an ultrasound transducer stack comprising one or more ultrasound transducers emitting an acoustic signal;
an acoustic lens focusing the acoustic signal; and
an acoustic coupling layer between the acoustic lens and the ultrasound transducer stack,
wherein the acoustic coupling layer has a speed of sound that is higher than a speed of sound in the acoustic lens, and
wherein the acoustic coupling layer has a thickness between a quarter and half a wavelength of the acoustic signal, and
wherein the acoustic lens includes at least one raised portion on the backside comprising at least one standoff that defines a space for the acoustic coupling layer between the acoustic lens and the ultrasound transducer stack.

2. The ultrasound probe of claim 1,
wherein the acoustic coupling layer has an acoustic impedance that substantially matches an acoustic impedance of the acoustic lens.

3. The ultrasound probe of claim 1,
wherein the thickness of the acoustic coupling layer is in a range of 75-200 micrometers (μm).

4. The ultrasound probe of claim 1,
wherein the acoustic coupling layer has an attenuation of 40-200 dB/cm at 5 MHz.

5. The ultrasound probe of claim 1,
wherein the acoustic coupling layer is made of a material selected from a group consisting of an epoxy and a silicone.

6. The ultrasound probe of claim 1,
wherein the acoustic coupling layer and the acoustic lens form an interface, and
wherein the interface impairs a transmission of a first acoustic wave from the acoustic lens into the acoustic coupling layer, wherein the first acoustic wave has an incident angle on the interface exceeding a critical angle.

7. The ultrasound probe of claim 6,
wherein the interface transmits a second acoustic wave from the acoustic lens into the acoustic coupling layer, wherein the second acoustic wave has an incident angle on the interface below the critical angle.

8. The ultrasound probe of claim 1,
wherein the one or more ultrasound transducers are at least one selected from a group consisting of a capacitive micromachined ultrasonic transducer (CMUT) and a piezoelectric micromachined ultrasonic transducer (PMUT).

9. (canceled)

10. The ultrasound probe of claim 19,
wherein the defined space is 0.090 mm+/−0.015 mm.

11. The ultrasound probe of claim 1,
wherein the acoustic lens is mechanically flexible.

12. An ultrasound probe, comprising:
an ultrasound transducer stack comprising one or more ultrasound transducers emitting an acoustic signal;
an acoustic lens focusing the acoustic signal; and
an acoustic coupling layer between the acoustic lens and the ultrasound transducer stack,
wherein the acoustic lens includes at least one raised portion on the backside comprising at least one standoff that defines a space for the acoustic coupling layer between the acoustic lens and the ultrasound transducer stack.

13. The ultrasound probe of claim 12,
wherein the defined space is 0.090 mm+/−0.015 mm.

14. The ultrasound probe of claim 12,
wherein the acoustic coupling layer has an acoustic impedance that substantially matches an acoustic impedance of the acoustic lens.

15. The ultrasound probe of claim 12,
wherein a thickness of the acoustic coupling layer is in a range of 75-200 micrometers (μm).

16. A method of manufacturing an ultrasound probe, the method comprising:
depositing an acoustic coupling layer between an ultrasound transducer stack comprising one or more ultrasound transducers for emitting an acoustic signal and an acoustic lens for focusing the acoustic signal,
wherein the acoustic lens comprises at least one standoff that defines a space for the acoustic coupling layer between the acoustic lens and the ultrasound transducer stack.

17. The method of claim 16, wherein depositing the acoustic coupling layer between the ultrasound transducer stack and the acoustic lens comprises:
depositing a liquid adhesive on one selected from a group consisting of the acoustic lens and the transducer stack;
joining the acoustic lens and the transducer stack, with the liquid adhesive between the acoustic lens and the transducer stack;
curing the liquid adhesive in the space defined by the at least one standoff to obtain the acoustic coupling layer.

18. The method of claim 17, wherein depositing the acoustic coupling layer between the ultrasound transducer stack and the acoustic lens further comprises:
deforming the acoustic lens as the ultrasound transducer stack and the acoustic lens are joined.

19. The method of claim 16, further comprising:
bonding the acoustic lens to a shroud, prior to depositing the acoustic coupling layer between the ultrasound transducer stack and the acoustic lens; and
securing the transducer stack to the shroud, after depositing the acoustic coupling layer between the ultrasound transducer stack and the acoustic lens.

20. The method of claim 16, further comprising:
installing the transducer stack in a shroud, prior to depositing the acoustic coupling layer between the ultrasound transducer stack and the acoustic lens; and
securing the acoustic lens to the shroud, after depositing the acoustic coupling layer between the ultrasound transducer stack and the acoustic lens.
